(19) United States
(12) Patent Application Publication
(10) Pub. No.: US 2007/0067029 A1
(43) Pub. Date: Mar. 22, 2007

(54) Support apparatus to facilitate implantation of cardiac prosthesis

(76) Inventor: Shlomo Gabbay, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/287,623

(22) Filed: Nov. 28, 2005

Related U.S. Application Data
(60) Provisional application No. 60/717,829, filed on Sep. 16, 2005.

(51) Int. Cl. A61F 2/24

(52) U.S. Cl. 623/2.13; 623/2.17; 623/2.38

(57) ABSTRACT

An apparatus includes substantially rigid and annular base portion having a radially outer sidewall portion and having a radially inner sidewall portion that defines an opening that extends axially through the base portion. A first rim portion extends substantially radially outwardly from the radially outer sidewall portion. A second rim portion extends substantially radially outwardly from the radially outer sidewall portion at an axial location that is spaced apart from the first rim portion so as to define a substantially continuous annular channel that extends circumferentially along the radially outer sidewall portion intermediate the first rim portion and second rim portion. The apparatus can be covered with a substantially biocompatible material.

### CROSS-REFERENCE TO RELATED APPLICATION

This application claims the benefit of provisional patent application 60/717,829, which was filed on Sep. 16, 2005, and entitled SYSTEM AND METHOD TO FACILITATE IMPLANTATION OF A VALVE, the entire contents of which is incorporated herein by reference.

### BACKGROUND

Various types of implantable prostheses have been developed and corresponding approaches are utilized to implant prostheses in both human and non-human patients. For example, it is known to utilize annuloplasty rings, stents other implantable cardiac prosthetic devices for helping improve functionality of a patient's heart valve. In more severe circumstances, implantable heart valve prostheses, such as natural tissue valves, mechanical valves and biomechanical valves are employed to replace a defective valve. In most cases, to surgically implant these and other cardiac prostheses into a patient's heart, the patient typically is placed on cardiopulmonary bypass during a complicated, but common, open chest and usually open-heart procedure. In an effort to reduce risk to the patient, minimally-invasive implantation techniques are continually being developed and improved. For instance, one type of minimally invasive technique utilizes a catheter that is advanced within the patient's body to the desired implantation site.

### SUMMARY

The present invention relates generally to a prosthesis or a support for a prosthesis that affords simplified implantation thereof For example, the prosthesis or support includes a base portion that permits use of a purse-string suture to connect the support or prosthesis at an annulus, such as a heart valve annulus. The support or prosthesis can be implemented in conjunction with virtually any type of cardiac prosthesis, including an annuloplasty ring apparatus, a valvuloplasty apparatus and a heart valve prosthesis (e.g., mechanical prosthesis, biological prosthesis or biomechanical prosthesis).

One aspect of the present invention provides an implantable apparatus that includes a substantially rigid and annular base portion having a radially outer sidewall portion and having a radially inner sidewall portion that defines an opening that extends axially through the base portion. A first rim portion extends substantially radially outwardly from the radially outer sidewall portion. A second rim portion extends substantially radially outwardly from the radially outer sidewall portion at an axial location that is spaced apart from the first rim portion so as to define a substantially continuous annular channel that extends circumferentially along the radially outer sidewall portion intermediate the first rim portion and second rim portion. The apparatus can be covered with a substantially biocompatible material (e.g., a natural material, a synthetic material, or combination of natural and synthetic materials).

Another aspect of the present invention provides a cardiac prosthesis that includes an annular base portion having inflow and outflow end portions spaced apart by a substantially annular sidewall portion that is configured to maintain a circumferential length thereof. An aperture extending axially through the base portion. A flexible covering of a substantially biocompatible material is connected over at least a radially outer surface of the sidewall portion. At least one feature that extends circumferentially around the sidewall portion and is covered by the covering to form at least one corresponding chamber that is defined by the covering, the at least one feature and by a portion the radially outer surface of the base portion.

The apparatus and/or prosthesis further can include additional structure operatively connected or extending from the base portion thereof, such as including a buttress member (e.g., for valvuloplasty) or a heart valve (e.g., for valve replacement).

### BRIEF DESCRIPTION OF THE DRAWINGS

FIG. 1 depicts an example of a ring apparatus according to an aspect of the present invention.

FIG. 2 depicts an example of the ring apparatus of FIG. 1 covered with a biocompatible material according to an aspect of the present invention.

FIG. 3 depicts another example of a ring apparatus according to an aspect of the present invention.

FIG. 4 depicts an isometric view of another apparatus that can be provided according to an aspect of the present invention.

FIG. 4A is a cross-sectional view take along line A-A through the base portion of the apparatus of FIG. 4.

FIG. 5 depicts another example of an apparatus that can be provided according to an aspect of the present invention.

FIG. 6 depicts an example of a ring apparatus that can be implemented according to an aspect of a present invention.

FIG. 7 depicts the ring apparatus of FIG. 6 covered with a biocompatible material according to an aspect of the present invention.

FIG. 8 depicts an example of a heart valve prosthesis within the apparatus of FIG. 7 according to an aspect of the present invention.

FIG. 9 depicts an alternative construction of an implantation apparatus according to an aspect of the present invention.

FIG. 10 depicts another example of a heart valve prosthesis that can be provided according to an aspect of the present invention.

FIG. 11 depicts yet another example of a heart valve prosthesis that can be provided according to an aspect of the present invention.

FIG. 12 depicts still another example of a heart valve prosthesis that can be provided according to an aspect of the present invention.

FIG. 13 depicts the heart valve prosthesis of FIG. 12 covered with a biocompatible material according to an aspect of the present invention.

FIG. 14 depicts a first portion of a procedure for implanting the apparatus of FIG. 2 according to an aspect of the present invention.

FIG. 15 depicts an example of a completed implantation of the apparatus of FIG. 2 according to an aspect of the present invention.

FIG. 16 depicts an example of the apparatus of FIG. 3 implanted at an annulus of a heart valve according to an aspect of the present invention.

FIG. 17 depicts a first portion of a procedure for implanting the apparatus of FIG. 5 at an annulus of a heart valve according to an aspect of the present invention.

FIG. 18 depicts a second portion of a procedure for implanting the apparatus of FIG. 5 according to an aspect of the present invention.

FIG. 19 depicts an example of a completed implantation of the prosthesis of FIG. 5 according to an aspect of the present invention.

FIG. 20 depicts an example of a heart valve prosthesis implanted at a first position in a patient's heart according to an aspect of the present invention.

FIG. 21 depicts an example of another heart valve prosthesis implanted at a second position in a patient's heart according to an aspect of the present invention.

### DETAILED DESCRIPTION

The present invention provides a prosthesis or a support for a prosthesis that facilitates implantation thereof For example, the prosthesis or support includes a base portion having a channel (or channel) in which a purse-string suture can be used to connect the support or prosthesis at an annulus, such as a valve annulus of a heart. The approach described herein can be utilized to implant various types of cardiac prostheses, including an annuloplasty ring apparatus, a valvuloplasty apparatus and a heart valve prosthesis (e.g., mechanical prosthesis, biological prosthesis or biomechanical prosthesis). As used herein, terms like “top”, “bottom”, “side” are utilized to depict relational views in the Figures shown, and are not intended to be of a limiting sense according to the position in which the device and apparatuses shown and described herein can be used.

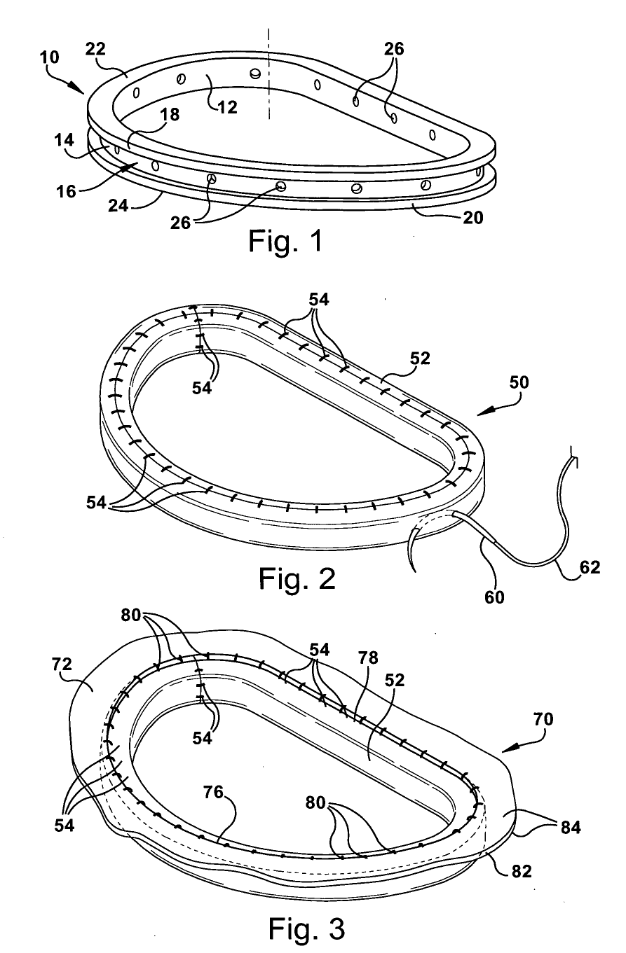

FIG. 1 depicts an example of a support apparatus 10, such as can be utilized as in annuloplasty reconstruction of a patient's heart valve. In the example of FIG. 1, the apparatus 10 is configured as a substantially D-shaped apparatus. It is to be understood that other shapes can also be utilized, including circular, elliptical, kidney-shaped and the like.

In the example of FIG. 1, the apparatus 10 provides a continuous substantially annular (or ring-like) base portion. The apparatus 10 includes a substantially continuous and substantially smooth interior surface 12. A radially outer sidewall portion 14 of the apparatus 10 includes a channel 16 extending circumferentially along at least a substantial portion the exterior surface. In the particular example of FIG. 1, the channel 16 is defined as the space that extends between axially spaced apart rim portions 18 and 20. That is, rim portions 18 and 20 protrude radially outwardly relative to the central portion of the outer sidewall portion 14 and are spaced apart from each other by an intermediate portion of the outer sidewall portion that provides the channel 16. For instance, the rim portions 18 and 20 can extend radially outwardly from respective top and bottom ends in a substantially parallel and opposing relationship to each other, which can be coextensive with the channel 16. This arrangement provides the apparatus 10 with a substantially U- or [-shaped cross section along the outer periphery of the apparatus.

While the channel 16 is shown and described as a substantially rectangular groove, it will be understood that the channel is not limited to such a configuration or shape. For example, the channel 16 can be implemented as a semicircular channel, dovetail channel or V-shaped channel to name a few. Additionally, more than one such channel can be provided (e.g., at axially spaced apart locations along the radially outer sidewall portion 14, but for purposes of simplicity a single channel is shown and described herein.

Also in the example of FIG. 1, axially opposed end surfaces 22 and 24 of the apparatus 10 are depicted as substantially flat planar surfaces that are substantially parallel to each other. It is to be understood and appreciated that the respective surfaces 22 and 24 need not be parallel to each other nor need they be substantially planar surfaces (e.g., surfaces 22 and 24 could be curved or beveled). Moreover, while the respective rim portions 18 and 20 are depicted as being of substantially the same size and configuration, the respective rim portions 18 and 20 and the surfaces 22 and 24 could be differently configured. For instance, one of the rim portions 18 or 20 can extend radially outwardly from the interior surface 12 of the apparatus 10 a distance that is greater than the radial extension of the other rim portion.

The apparatus 10 can also include a plurality of apertures 26 that extend from the interior sidewall portion 12 through to the exterior portion 14 along the channel 16. That is, the apertures 26 are positioned at an intermediate position between the top and bottom end surfaces 22 and 24 of the apparatus 10. The apertures 26 can be spaced substantially evenly along the periphery of the apparatus 10 to facilitate attachment of a substantially biocompatible material covering, such as shown in FIG. 2.

The apparatus 10 can be formed of any substantially rigid material, such as metal (e.g., surgical grade stainless steel or an alloy thereof) or plastic (e.g., Delrin). By “substantially rigid,” it is meant that the material is designed to maintain its original form, although it may be exhibit flexibility and resilience when force is applied. For example, the apparatus 10 can also be compliant such that it is deformable under force and resiliently returns to its substantially original form when the force is removed. Those skilled in the art will understand and appreciate various manufacturing techniques that can be employed to make the apparatus, including injection molding, stamping, casting, to name a few. The apparatus 10 is not limited to any of method of manufacture, however. The base portion of the apparatus 10 can be configured to maintain a circumferential length thereof. By maintaining a circumferential length, it is meant that the radially outer sidewall portion of the apparatus keeps a substantially constant length, even if it is deformed (e.g., by radial or torsional forces).

The rim portions 18 and 20 can be monolithic with the base portion from which they extend or the rim portions may be attached to the base portion. As used herein, the term “monolithic” means that the structural portions of the apparatus are fabricated as a single structure (e.g., integrally formed as a single piece), although the materials that form different portions of such structure can be the same or different materials.

As depicted in FIG. 2, the apparatus 10 can be covered with a biocompatible material to provide a covered apparatus 50. In the example of FIG. 2, the apparatus 50 is completely covered, including the radially interior surface 12, the radially exterior surface 14 and the axial (e.g., inflow and outflow end) surfaces 22 and 24. The biocompatible material, for example, can be a biological tissue material (e.g., animal pericardium, collagen, dura matter and the like), a synthetic material, (e.g., Dacron or other fabric material), or a naturally occurring fabric material, such as cotton or other textiles as well as any combination of materials. The covering 52 can be applied as one or more sheets of the biocompatible material or as a coating of a suitable biocompatible material that is applied onto the apparatus 10, such as by attaching opposing end portions by sutures 54. The above examples are not to be limiting of the type of biocompatible material that can be utilized to form the covered apparatus 50 but are merely intended to provide examples of possible known materials that can be used.

By way of further example, the covering 52 may be formed from one or more sheets of a NO-REACT® tissue product, which are commercially available from Shelhigh, Inc., of Millburn, N.J. The NO-REACT® tissue products help improve the biocompatibility of the apparatus 50, thereby mitigating the likelihood of a patient rejecting an implanted prosthesis that includes the apparatus. The NO-REACT® tissue also has been shown to resist calcification when implanted in vivo.

As depicted in FIG. 2, the covering 52 extends over at least the radially outer portion 14 of the apparatus 10 so that a circumferentially extending chamber (or space) is formed between the covering and a portion of radially outer surface 14. In the example of FIG. 2, a substantially annular chamber is defined by adjacent surfaces of the covering 52 and the channel 16. By covering the annular channel 16 with the outer covering 52, implantation is facilitated. For instance, a needle 60 and a length of suture (indicated at 62) can be inserted through the outer covering 52 into the channel (or chamber) 16 for securing the prosthesis at a desired implantation site via purse string suture. The chamber may be substantially hollow or, alternatively, the chamber may contain one or more materials, such as a substantially soft or flexible (e.g., natural or synthetic) material, such as the material utilized to provide the covering 52.

FIG. 3 depicts another example of a covered apparatus 70 that can be implemented according to an aspect of the present invention. The apparatus 70 is substantially similar to that shown and described in FIG. 2, in which the same reference numbers refer to corresponding structure previously shown and described with respect to FIGS. 1 and 2.

The apparatus 70 includes a flange 72 that extends substantially radially outwardly from one of the ends 74 of the apparatus 70. The flange 72 can be formed from one or more sheets of a substantially flexible sheet of substantially biocompatible material. The. flange 72 can be formed of the same or a different material from the covering 52 that is applied over the support apparatus 10. As one example, the flange 72 is formed of one or more sheets of material having a radially inner edge (or periphery) 76 that is attached at the inflow end of the apparatus 78, such as by sutures 80. The flange 72 also includes a radially outer edge 82 that is spaced apart from the inner edge 76 by top and bottom opposing surfaces 84. For instance, the flange 72 can be formed of a pair of sheets of the biocompatible material that are attached together. When the flange 72 includes more than one sheet of material, the sheets can be attached together at the time of attachment to the apparatus or at a time prior thereto, such as by suturing the radially inner edges and radially outer edges thereof together. As shown and described herein, the flange 72 can be positioned in an overlying relationship at the annulus at which the apparatus 70 is implanted to cover exposed sutures at the implantation site. The flange 72 also can improve hemodynamics at the inflow end of the apparatus 70 since the flange provides a substantially continuous and smooth transition from the adjacent tissue into the apparatus.

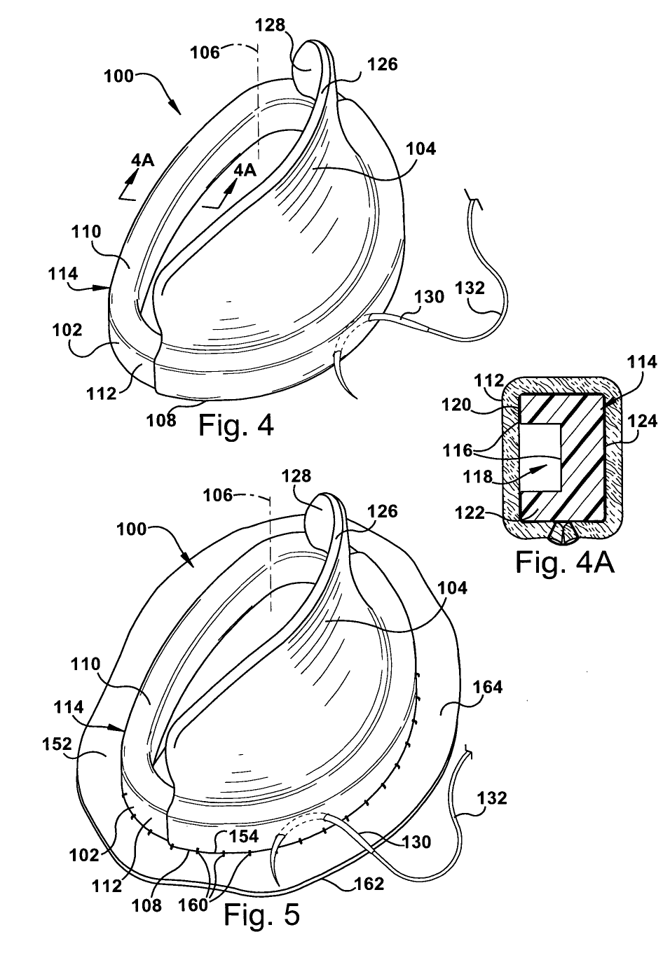

FIG. 4 depicts example of another type of support apparatus according to an aspect of the present invention. The apparatus 100 includes a base portion 102 and a buttress member 104 that extends generally axially from a portion of the base portion.

The base portion 102, which may be an oval shape, egg-shaped or another suitable shape dimensioned and configured for attachment at an annulus of a heart valve. A central axis 106 extends through the apparatus 100 substantially transverse to a plane extending through the base portion 102. The base portion, however, need no be planar (e.g., it could have a substantially sinusoidal contour). The base portion 102 has an inflow end 108 and an outflow end 110.

In the example of FIG. 4, the base portion includes a covering 112 of a substantially biocompatible material over a substantially annular support 114. The covering 112 can be implemented as one or more sheets or layers of a biocompatible material, such as those described herein. The support 114 can be formed a substantially resilient material, such as metal or plastic, that is designed to provide a desired amount of rigidity and flexibility for supporting an annulus of a heart valve. For instance, the support 114 can be made of a medical grade polymer that is compliant but sufficient resilient to return to substantially its original configuration after being deformed. The support 114 provides the benefits of an annuloplasty ring (e.g., it helps support a valve annulus at a desired orientation at systole). The rigidity or flexibility of the support 114 may vary depending upon the amount of support desired at the annulus.

The support includes a radially exterior surface 116 having a channel or channel 118 along at least a substantial portion of the exterior surface. In the example of FIG. 1, the channel 16 is defined as the space that extends between top and bottom of protruding rim portions 120 and 122. The radially inner sidewall 124 surface of the support 114 can be substantially smooth and continuous. That is, the top and bottom surfaces support include the rim portions 120 and 122 that extend radially outwardly from respective top and bottom ends of the support, such as to provide a generally C-shaped cross section along the periphery of the support 114. Those skilled in the art will understand and appreciate other configurations that can be implemented at the exterior surface 116 to provide a corresponding channel along the exterior surface. The support 114 further can include apertures (not shown) that extend through the sidewall for use in securing the covering 112 to the support.

The buttress member 104 that is attached to and extends from the base portion 102 and terminates in an outflow end 126. The buttress member 104 has a radially inner surface that is dimensioned and configured to approximate (or simulate) the dimensions and configuration of one or more heart valve leaflets in a closed position. As depicted in FIG. 4, for instance, the buttress member 104 extends radially inwardly and axially from a predetermined circumferentially extending arc length of the base portion 102. The arc length of the base portion 102 from which the buttress member 104 extends can vary (e.g., from about 90 degrees to about 270 degrees) according to size and type of valve where the apparatus is intended to be implanted. By way of example, when the apparatus 100 is to be implanted at the annulus of a mitral valve and function in place of a posterior leaflet, the circumferential arc of the buttress may approximate the arc length of the annulus adjacent the posterior leaflet of the patient's valve. That is, a radially inner surface 128 of the buttress 104 can be dimensioned and configured to substantially maintain the shape of a closed posterior leaflet so as to provide a surface against which the patient's anterior leaflet may coapt.

The buttress 104 can also be covered with a biocompatible material, which may be the same material that covers the support 114. For instance, the buttress 104 can be formed of one or more sheets of biocompatible material that are attached to and extend from the base portion 102. Alternatively or additionally, the buttress 104 can include an underlying support structure that is covered with the biocompatible material. Such support structure can include one or more support elements that extend from the support 114. Such one or more support elements can be monolithic or attached to the annular support 114. The rigidity or flexibility of the buttress 104 may vary depending upon the amount of flexibility desired during engagement between a patient's leaflet(s) and the inwardly exposed surface 128 of the buttress 104.

In the example of the apparatus 100 shown in FIG. 4 (having a complete and continuous annular base portion 102), an aperture extends axially through the apparatus 100 between another arc length of the base portion 102 and the buttress 104 itself The aperture provides an opening or orifice to permit the passage of blood through the apparatus 100, such as during diastole. The buttress 104 in conjunction with the leaflet (or leaflets) of a patient's native heart valve inhibits the flow of blood when the valve is in a closed position, such as during ventricular contraction at systole. The radially inner surface 128 of the buttress 104 can thus provide a substantially fixed structure against which a leaflet of a heart valve may engage and substantially coapt with at diastole. By “substantially fixed” structure, it is meant that the buttress substantially maintains the dimensions and configuration of one or more heart valve leaflets in the closed position, although the buttress can be sufficiently flexible to allow some movement as blood flows through the aperture or when engaged by an adjacent leaflet. The amount of movement generally depends on whether any support structure(s) or substrate materials are utilized to form the buttress. Further examples of buttress structures that can be implemented in the apparatus 100 are disclosed in U.S. Pat. No. 6,419,695, which is incorporated herein by reference.

As depicted in FIG. 4, the covering 112 extends over at least the radially outer surface 116 of the apparatus 10 so that a circumferentially extending chamber (or space) is formed between the covering and a portion of radially outer surface 116. In the example of FIG. 4, a substantially annular chamber is defined by adjacent surfaces of the covering 112 and the channel 118. By covering the annular channel 118 with the outer covering 52 that is affixed to the support, implantation is facilitated. For instance, a needle 130 and a length of suture 132 can be inserted through the outer covering 112 into the channel 118 for securing the prosthesis at a desired implantation site, such as described herein. The resulting chamber may be substantially hollow or, alternatively, the chamber may contain a substantially soft or flexible (e.g., natural or synthetic) material, such as the material utilized to provide the covering 112.

FIG. 5 depicts another example of a valvuloplasty apparatus 150 that can be implemented according to an aspect of the present invention. For purposes of simplicity of explanation regarding, the same reference numbers are used in FIG. 5 to refer to corresponding structure previously shown and described with respect to FIG. 4.

The apparatus 150 includes an implantation flange 152 that extends radially outwardly from the base portion 102. The flange 152 can extend from the inflow end 108 of the base portion 102. Alternatively, the flange 152 may extend from the other end 110 of the base portion 102.

The flange 152 can be formed from one or more sheets of a substantially flexible sheet of substantially biocompatible material, such as described herein. The flange 152 can be formed of the same or a different material from the covering 112 that is applied over the support 114. As one example, the implantation flange 126 is formed of a substantially biocompatible biological material, such as animal tissue (e.g., animal pericardium, dura matter or the like). The implantation flange 152 may be formed as an integral part of the covering 112 or as a separate structure that is attached to the base portion 102, such as by sutures.

As one example, the flange 152 is formed of one or more sheets of material having a radially inner edge (or periphery) 156 that is attached at or adjacent the inflow end 108 of the base portion 102, such as by sutures 160. The flange 152 also includes a radially outer edge 162 that is spaced apart from the radially inner edge 156 thereof by opposing surfaces 164. As another example, the flange 152 can be formed of a pair of sheets of the biocompatible material that are attached together. When the flange 152 includes more than one sheet of material, the sheets can be attached together at the time of attachment to the apparatus or at a time prior thereto, such as by suturing the radially inner edges and radially outer edges thereof together.

As shown and described herein, the flange 152 can be positioned in an overlying relationship at the annulus at which the apparatus 150 is implanted to cover exposed sutures at the implantation site. The flange 152 can also improve hemodynamics of the apparatus 150 at the inflow end of the apparatus 150 since the flange 152 provides a substantially continuous and smooth transition for blood flow from the adjacent tissue into the apparatus.

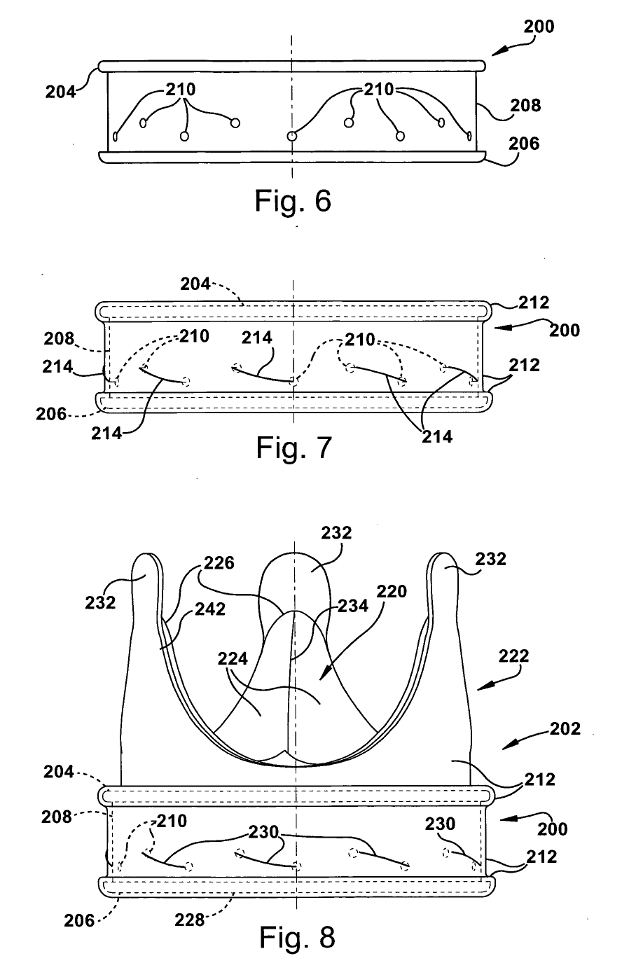

FIGS. 6 and 7 depict another example of an implantation apparatus 200. As shown in FIG. 6, the implantation apparatus 200 includes a pair of rims 204 and 206 that are axially spaced apart from each other by a generally cylindrical sidewall 208. The axial length of the sidewall 208 can vary according to the type of cardiac prosthesis that is mounted therein for implantation. Additionally, while the sidewall portion 208 is shown as a generally smooth sidewall, the sidewall portion could be curved radially inwardly, radially outwardly or both along its axial length.

Each of the rims 204 and 208 extend radially outwardly beyond the radially outer surface of the sidewall 208 so as to define a channel between the respective rims. Each of the rims 204 and 208 can be formed of the same or different materials and have a thickness (in the axial direction) to provide a desired amount of flexibility or rigidity for the apparatus 200. An appropriate amount of flexibility or rigidity may vary, for example, depending on the amount of support that may be needed at a patient's annulus where the apparatus is to be implanted. For example, the rim 206 corresponds to an inflow flange and, thus, can have a curved cross-sectional contour (e.g., curving axially toward the other flange 204) to provide for improved hemodynamics when implanted in a patient's heart.

The sidewall 208 can also include a plurality of apertures 210 that extend through the sidewall 208 at circumferentially spaced apart locations about the sidewall. The apertures 210 extend through the sidewall 208 at an axial location that is substantially intermediate ends of the sidewall 208. As one example, the apertures 210 can be arranged as two (or more) axially and circumferentially spaced apart rows of apertures. Other arrangements and configurations of apertures 210 can also be utilized.

As depicted in FIG. 7, the implantation apparatus 200 includes a covering 212 of a substantially biocompatible material 212, such as any of the biocompatible materials described herein. For example, the covering 212 can include one or more sheets of biocompatible material that are applied to cover all exposed surfaces of the implantation apparatus 200, which can include interior as well as exterior surfaces. One or more sutures 214 can be applied through the apertures 210 to secure the covering 212 relative to the implantation apparatus 200. Additional sutures 214 can be applied near the radially outer peripheries of the respective rims 204 and 206 and at the juncture near the junctures of the sidewall 208 and the rims to secure the biocompatible material 212 relative to the various surfaces of the implantation apparatus 200. Additionally and alternatively, other means for securing the outer covering 212 to the surfaces of the apparatus 200 can be utilized, such as adhesives, clamps and/or other arrangements of one or more sutures. As described herein, multiple layers (e.g., of one or more sheets) of the biocompatible material 212 can be provided around the sidewall 208 to facilitate implantation using a purse-string suture, which can be applied into and through the one or more of the layers of the biocompatible material 212. In FIG. 7, the covering 212 is depicted as being substantially flush with the radially outer surface of the sidewall 208. It is to be understood that, alternatively, the covering can be applied as an extension that directly connects the radially outer edges rim portions 204 and 206. This alternative configuration provides a space between the covering 212 and the sidewall corresponding to the channel into which a needle and suture may be passed into and out of repeatedly to facilitate attachment of the implantation apparatus 200 during purse string implantation.

In FIG. 8, a heart valve 220 has been attached to the implantation apparatus 200 of FIG. 7 to provide a heart valve prosthesis 222 according to an aspect of the present invention. The heart valve 220 can be any type of heart valve, including a natural tissue valve (e.g., an animal heart valve or a valve manufactured from biological tissue), a mechanical valve, or a biomechanical valve. The valve 220 may be stented or unstented, depending generally on the type of valve and the amount of support needed at the implantation site. For instance, the apparatus 200 will provide support at an annulus to help maintain the annulus in a desired configuration according to the configuration of the apparatus 200. Other types of cardiac prostheses, including known and yet to be developed devices, can also be mounted within the apparatus 200.

In the example of FIG. 8, the valve 220 includes one or more moveable members that operate to provide for substantially unidirectional flow of blood. For the particular natural tissue heart valve depicted in the example of FIG. 8, the valve 220 includes a plurality of leaflets 224 that are moveable relative to each other (e.g., between open and closed positions) to provide for the substantially unidirectional flow of blood through the prosthesis 222. The valve 220 includes an outflow end 226 that extends axially beyond the rim 204. An inflow end of the valve 220 is located near the other flange 206, such as may be coextensive with the inflow end 228 of the apparatus 200. For example, sutures 230 can be applied to some or all of the apertures 210 to secure the valve 220 relative to the sidewall of the implantation apparatus 200.

The prosthesis 222 may also include outflow lobes (or extension) 232 located at an outflow end 226 of the prosthesis, such as when the valve 220 is a natural tissue valve (e.g., an animal heterograft or manufactured valve). The lobes are provided at a location near respective commissures 234 between adjacent pairs of leaflets 224. The lobes 232 extend a predetermined distance beyond and lateral to each of the commissures 234 at the outflow end 226 of the valve 220. The surgeon implanting the prosthesis 222 can cut the lobes 232 to a desired shape and size. The particular size of the lobes 232 also will depend upon the size of the prosthesis 222. Intermediate each adjacent pair of lobes 232, the outflow end of the outer covering 212 can follow the contour of the outflow end 226 of the valve 220 (e.g., generally sinusoidal outflow end). The lobes 232 can be part of the outer covering 212, such as shown in FIG. 8. Alternatively, the lobes 232 can be separate sheets of biocompatible material (e.g., natural or synthetic) attached to the outer surface of the valve 220 at the respective commissures 224 proximal the outflow end of the valve. The lobes 232 thus provide extensions at the outflow end commissures 224, which can be secured to a patient's tissue (e.g., the patient's valve wall).

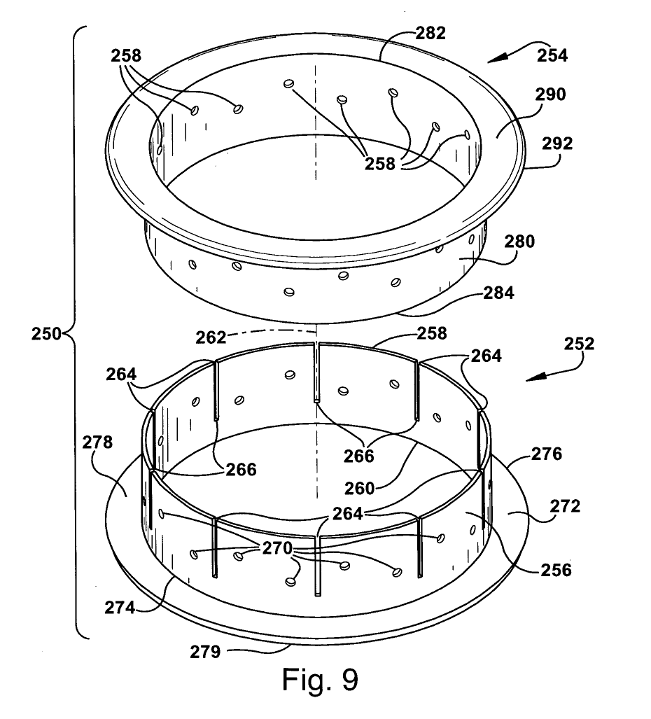

FIG. 9 depicts an exploded view of another configuration of an implantation apparatus 250 that can be implemented according to an aspect of the present invention. The apparatus 250 includes separate first and second prosthesis portions 252 and 254, respectively. In the assembled condition, the first and second prosthesis portions 252 and 254 form an implantation apparatus that is substantially similar to that shown and described with respect to FIG. 6. It is further to be understood that the implantation apparatus 250 can be utilized to provide a means for implanting various types of cardiac prosthesis, including heart valves such as biological or natural tissue heart valves, mechanical heart valves and biomechanical heart valves.

The first prosthesis portion 252 includes a generally cylindrical sidewall portion 256 that extends from a proximal end 258 and terminates in a distal end 260. The axial length of the sidewall portion 256 can vary according to desired sizing requirements of the apparatus 250. As an example, the length of the first prosthesis portion 252 can be provided at different axial lengths, such as ranging from about 5 millimeters to about 15 millimeters. The cylindrical sidewall portion 256 extends generally parallel to a central axis 262 that extends through the first prosthesis portion 252.

While in the example of FIG. 9, the first prosthesis portion 254 is depicted as having a substantially right circular cylindrical sidewall portion 12, it is to be understood and appreciated that the sidewall portion 12 is not limited to such a shape. For example, the sidewall 256 can be substantially frusto-conical, or have an elliptical cross section, a D-shaped cross-section, or other shapes depending upon the desired configuration of the resulting prosthesis.

A plurality of notches or slots 264 extend from the proximal end 258 of the sidewall portion 256 and terminate at an axial location 266 on the sidewall portion that is intermediate the proximal end 258 and the distal end 260. Since the slots 264 terminate at the location 266, at least a portion of the sidewall 256 forms a continuous annular band that will maintain a substantially fixed circumferential dimension. By maintaining a circumferential dimension, the sidewall 256 keeps a substantially constant length along at least a portion of its perimeter, even if the sidewall is deformed radially. The longitudinally extending slots 264 permit the sidewall portion to flex radially and more easily permit the similarly dimensioned second prosthesis portion 254 to fit at least partially within the sidewall portion 256. Additionally or alternatively, the slots 264 permit the sidewall portion 256 to fit at least partially into the similarly dimensioned and configured second prosthesis portion 254.

The first prosthesis portion 252 can also include a plurality of apertures 270 that extend through the sidewall portion 256 at circumferentially spaced apart locations about the sidewall portion. As shown in FIG. 9, the apertures 270 extend through the sidewall portion 256 substantially intermediate the proximal and distal ends 258 and 260. As one example, the apertures 270 can be arranged as two (or more) axially spaced apart rows of such apertures. Additionally, a pair of respective apertures 270 can be provided between adjacent pairs of the slots 264. The apertures 270 can be utilized to attach a biocompatible covering to the first prosthesis portion 10, such as by being dimensioned to permit a needle and suture to pass substantially freely there through the respective apertures.

The first prosthesis portion 252 can also includes a rim 272. The rim 272 extends radially outwardly from the distal end 260 of the sidewall portion 256. As one example, the rim 272 has an inner periphery 274 that is coextensive with the distal end 260 of the sidewall portion 256. The rim 272 and the sidewall portion 256 can be a monolithic (or single piece) construction. The rim 272 is defined by opposing surfaces 278 and 279 that extend from the inner periphery 274 to an outer periphery 276 thereof In one example, at least the side surface 279 of the rim 272 opposite the sidewall portion 256 is curved radially and axially toward the proximal end 258. In this configuration, the rim 272 extends arcuately a distance axially from the distal end 260 and toward the proximal end 258 of the sidewall portion 256. The curved rim 272 of the first prosthesis portion 252 can help provide for improved hemodynamics when implanted at heart valve annulus (e.g., at an inflow side of the valve). Alternatively, the surface 279 of the rim 272 can be substantially planar or have other curved contours.

The first prosthesis portion 252 further can be covered with a biocompatible material, such as described herein. The covering can include one or more sheets or layers of the biocompatible material. To help secure the biocompatible material to the prosthesis portion 252, one or more sutures can be applied through the apertures 270. Additionally and alternatively, other means for securing the biocompatible covering to the prosthesis portion 10 can be utilized, such as adhesives, clamps or other arrangements of one or more sutures. The covering may also be applied in other manners (e.g., spraying or chemically bonding one or more layers of a suitable material).

The second prosthesis portion 254 can be attached to first prosthesis portion 252 to form an implantation apparatus 250 similar in function to the apparatus 200 shown and described in FIGS. 6-8. The second prosthesis portion 254 includes a substantially cylindrical sidewall portion 280 that extends from a proximal end 282 and terminates in a distal end 284 that is spaced apart from the proximal end a distance. The axial length of the second prosthesis portion 254 can have varying different dimensions, such as depending upon the dimensions of the first prosthesis portion 252, the size of the prosthesis to be mounted within the apparatus 250 and patient's anatomy, for example.

The sidewall portion 280 is a substantially cylindrical sidewall (e.g., a right cylindrical sidewall), although it is not limited to such a configuration. As described above, the sidewall portion 280 can be dimensioned and configured to be inserted within the sidewall portion 256 of the first prosthesis portion 252. Alternatively, the second prosthesis portion 254 can be dimensioned and configured to receive the proximal sidewall portion of the first prosthesis portion therein. For instance, the second prosthesis portion 254 can include slots (not shown) in its sidewall 280 similar to the slots 264 of the first prosthesis portion 252.

In the example where the sidewall portion 280 is to be received within the proximal portion of the sidewall portion 256 of the first prosthesis portion 252, the outer diameter of the sidewall portion 280 (at least near the distal end 284) can be configured to be substantially equal to or slightly larger than the inner diameter of the sidewall portion 256. For example, the outer diameter of the sidewall portion 280 of the second prosthesis portion may be approximately 0.2 to about 0.5 millimeters greater than the inner diameter of the sidewall portion 256 of the first prosthesis portion 252, such as where a friction fitting is to be utilized to hold the prosthesis portions together.

Similar to the first prosthesis portion, the second prosthesis portion 254 also includes a plurality of apertures 288 formed through the sidewall portion 280. The diameter of the apertures 288 can be similar to the diameter of the apertures 270 of the first prosthesis portion 252, such that they can facilitate suturing an outer covering onto the second prosthesis portion (see, e.g., FIG. 7). The apertures 288 are spaced apart circumferentially about the sidewall portion 280, such as in the arrangement as shown in FIG. 9 in which a first set of apertures is at a first axial dimension closer to the distal end 284 and a second set of apertures is closer to the proximal end 282 of the sidewall portion 280.

The second prosthesis portion 254 also includes a rim 290 that extends outwardly from the sidewall portion 280 adjacent the proximal end 282 thereof. The rim 290 can be formed with the sidewall portion as a monolithic structure. The rim 290 can have an axial thickness to provide a desired amount of rigidity and flexibility to the second prosthesis portion 254. A radially outer extent 292 of the rim 290 can be curved between top and bottom opposing surfaces of the rim 290, respectively, thereby providing a curved surface corresponding to the axial thickness of the rim between the opposing surfaces thereof

In the example of FIG. 9, the rim 290 is configured as an annular disc having a substantially flat proximal surface 294 and a substantially flat lower surface; the distance between the opposing surfaces of the rim corresponding to the thickness of the rim 290. An inner diameter of the second prosthesis portion 254 can substantially correspond to the outer diameter of a heart valve (e.g., natural tissue, mechanical or biomechanical) that is to be mounted within the second prosthesis portion. As described with respect to the first prosthesis portion 252, the second prosthesis portion 254 can be covered with a substantially biocompatible material to provide a corresponding covered second prosthesis. Those skilled in the art will understand and appreciate various materials and coatings that could be utilized to provide such a covering for the prosthesis portions based on the teachings contained herein. Thus, when the covered prosthesis portions are attached together, a support structure for implanting a cardiac prosthesis is provided, such as similar to that shown in FIG. 7.

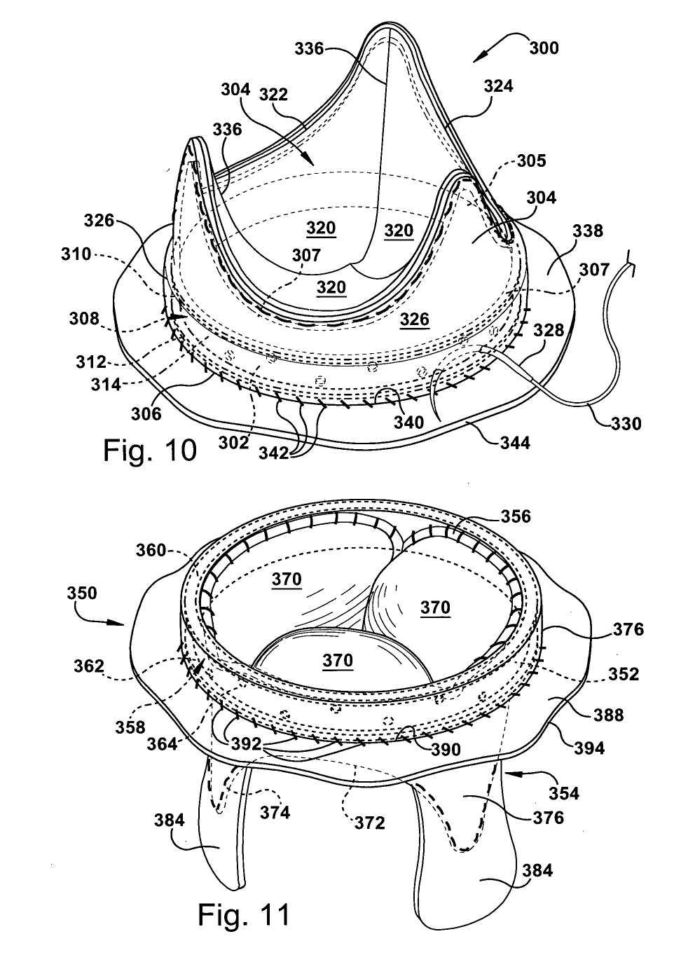

FIG. 10 depicts an example of another heart valve prosthesis 300 that can be implemented according to an aspect of the present invention. The prosthesis 300 includes an implantation apparatus 302 and a heart valve 304 mounted within the apparatus. In the example of FIG. 10, the heart valve includes a natural tissue valve member 304 mounted within a stent 305 (e.g., corresponding to a fully stented valve). It is to be understood that the valve can be stented or unstented. Additionally or alternatively, it is to be understood that the valve 304 could be implemented as a different type of natural tissue valve, a mechanical valve or biomechanical valve. In the example shown in FIG. 10, the stent 305 includes an inflow end and an outflow end 307 that are substantially co-extensive with the inflow and outflow ends of the valve 304. For instance, the outflow end 307 of the stent 305 is substantially sinusoidal, having stent posts aligned substantially radially with commissures 336 of the valve 304.

The apparatus 302 is positioned around the stent 305 and the valve 304 at a position such that the apparatus is adjacent the inflow end 306 of the valve and the inflow end of the stent. The implantation apparatus 302 includes a substantially annular channel 308 that extends circumferentially around the perimeter of the apparatus. The annular channel 308 is provided as a space between axially spaced apart rim portions 310 and 312, which extend radially outwardly from a central sidewall portion 314 of the implantation apparatus 302. The rim portions 310 and 312 extend radially outwardly beyond the exterior surface of the central sidewall portion 314, for example, a distance that approximates or is greater than the diameter of a surgical needle that is to be used to implant the prosthesis 300. While the exterior surface of the central sidewall portion 314 is depicted as being a substantially right circular cylinder, it is to be understood that the surface could be curved.

The valve 304 can be attached to the implantation apparatus 302 such as by sutures. It will be appreciated that other means for attaching the valve 304 and/or the stent 305 relative to the apparatus 302 can also be utilized, including adhesives, clamps, barbs, friction, latches and the like, which may vary according to the type and configuration of valve and stent being utilized. As discussed above, the valve 304 can be any type of heart valve.

By way of further example, the valve 304 is depicted as a natural tissue heart valve, namely an animal heart valve. The valve 304 includes a plurality of leaflets 320 that moveable relative to each other (e.g., between open and closed positions) to provide for the substantially unidirectional flow of blood through the prosthesis 300. For the animal-type heart valve shown in FIG. 10, the leaflets 320 are mounted within a portion of valve wall 322, which has been trimmed to provide a substantially sinusoidal outflow end portion 324. The outflow end portion 324 extends axially beyond the rim 310. The inflow end of the valve 304 and stent 305 are located adjacent the other rim 312, such as may be coextensive with the inflow end 306 of the apparatus 302.

An outer covering 326 of a substantially biocompatible material covers the implantation apparatus 302 and at least a substantial portion of the stent 305 and the valve 304. The covering 326 can be selected as the type of biocompatible materials described herein, although it is not limited to such materials. The covering 326 covers the radially outer surface of the stent 305. The covering 326 can be attached to the valve 304, such as by one or more sutures (e.g., continuous or uninterrupted sutures) that are applied at the outflow end of the stented valve. The covering thus can help secure the valve 304 to the stent 305.

The covering 326 also extends over the radially outer portion of the implantation apparatus 302 so that a chamber is formed by the channel 308 and the covering. That is, a substantially annular chamber is defined by adjacent surfaces of the covering 326, the sidewall portion 314 and the rim portions 310 and 312. The chamber may be substantially hollow or, alternatively, the chamber may contain a substantially soft or flexible (e.g., natural or synthetic) material. The fill material may be the same or a different biocompatible material from the material utilized to provide the covering 326.

By covering the annular channel 308 with the outer covering 326, implantation is facilitated. For instance, a needle 328 and a length of suture (indicated at 330) can be inserted through the outer covering 326 into the channel 308 for securing the prosthesis 300 at a desired implantation site via purse string suture. Thus, as the suture 330 is tightened around the annular channel 308 and against the corresponding outer sidewall portion 314 of the base 302, axial movement of the prosthesis 300 will be mitigated since the suture will be retained between the rim portions.

The prosthesis 300 also includes a flange 338 that extends radially outwardly from the implantation apparatus 302 of the prosthesis adjacent the inflow edge of the prosthesis. For example, the flange 338 has a radially inner periphery or edge 340 that is attached (e.g., by sutures 342) to the outer covering 326 adjacent to the rim portion 312. This flange position is well suited for use in implanting the prosthesis at an mitral position of a patient's heart; although, the prosthesis 350 is not limited to use for replacing a mitral valve. The flange 338 extends from the inner edge 340 radially outwardly a predetermined distance and terminates in an outer periphery or edge 344. The flange 338 can be formed of any substantially flexible and biocompatible material, such as a natural or synthetic material described herein. As one example, the flange 338 can be formed of one or more sheets of animal pericardium. When more than one sheets of such material is utilized to form the flange 338, the radially outer edge 344 of the sheets can be attached together, such as by one or more continuous sutures at one or both of the inner edge 340 and the outer edge 344.

FIG. 11 depicts an alternative example of a heart valve prosthesis 350 that can be provided according to an aspect of the present invention. The prosthesis 350 includes an implantation apparatus 352 and a heart valve 354 mounted within the apparatus. In the example of FIG. 10, the heart valve 354 includes a natural tissue valve member mounted within the apparatus 352. It is to be understood that the valve 304 may be stented or unstented. Additionally or alternatively, it is to be understood that the valve 354 could be implemented as a different type of natural tissue valve, a mechanical valve or biomechanical valve.

The apparatus 352 is positioned around the valve 354 at a position that is adjacent an inflow end 356 of the valve. The implantation apparatus 352 includes a substantially annular channel 358 that extends circumferentially around the perimeter of the apparatus and circumscribes the inflow portion of the valve 304. The annular channel 358 is provided as a space between axially spaced apart rim portions 360 and 362, which extend radially outwardly from a central sidewall portion 364 of the implantation apparatus 352. The rim portions 360 and 362 extend radially outwardly beyond the exterior surface of the central sidewall portion 364, for example, a distance that approximates or is greater than the diameter of a surgical needle that is to be used to implant the prosthesis 350. While the exterior surface of the central sidewall portion 364 is depicted as being a substantially right circular cylinder, it is to be understood that the surface could be curved or have other shapes, such as oval, elliptical, or substantially D-shaped to name a few.

The valve 354 can be attached to the implantation apparatus 352 such as by sutures (not shown). For example, such sutures can be applied to the inflow end portion of the valve via a series of one or more apertures that extend through the sidewall portion 364 of the apparatus. It will be appreciated that other means for attaching the valve 354 relative to the apparatus 352 can also be utilized, including adhesives, clamps, barbs, friction, and the like, which may vary according to the type of valve being utilized.

By way of example, the valve 354 is depicted as a natural tissue heart valve, such as an animal (e.g., bovine, equine or porcine) heart valve. The valve 354 includes a plurality of leaflets 370 that moveable relative to each other (e.g., between open and closed positions) to provide for the substantially unidirectional flow of blood through the prosthesis 350. For the type of animal heart valve shown in FIG. 10, the leaflets 370 are mounted within a portion of valve wall 372, which has been trimmed to provide a substantially sinusoidal outflow end portion 374. The valve 304 is not limited to such a valve, however. The outflow end portion 374 of the valve 335 extends axially beyond the rim 362. The inflow end of the valve 356 is located adjacent the other rim 306, such as may be coextensive with the inflow end 356 of the apparatus 352.

An outer covering 376 of a substantially biocompatible material covers the implantation apparatus 352 and at least a substantial portion of the valve 354. The covering 376 can be selected as the type of biocompatible materials described herein, although it is not limited to such materials. The covering 376 extends over the radially outer portion of the implantation apparatus 352 so that a chamber is formed by the channel 358 and the covering. That is, a substantially annular chamber is defined by adjacent surfaces of the covering 376, the sidewall portion 364 and the rim portions 360 and 362. The chamber may be substantially hollow or, alternatively, the chamber may contain a substantially soft or flexible (e.g., natural or synthetic) fill material (not shown). The fill material may be the same or a different biocompatible material from the material utilized to provide the covering 376.

By covering the annular channel 358 with the outer covering 376, implantation is facilitated. For instance, a needle 378 and a length of suture (indicated at 380) can be inserted through the outer covering 376 into the channel 358 for securing the prosthesis 350 at a desired implantation site via purse string suture. Thus, as the suture 380 is tightened around the annular channel 358 and against the corresponding outer sidewall portion 364 of the base 352, axial movement of the prosthesis 350 will be mitigated since the suture will be retained between the rim portions.

The prosthesis 350 may also include outflow lobes (or extension) 384 located at an outflow end 374 of the valve 354 near each of the commissures 386 between adjacent pairs of leaflets. The lobes 384 extend a predetermined distance beyond and lateral to each of the commissures 386 at the outflow end 374 of the valve 354. The surgeon implanting the prosthesis 350 might thus cut the lobes 384 to a desired shape and size. The particular size of the lobes 384 also will depend upon the size of the prosthesis 350. Intermediate each adjacent pair of lobes 384, the outflow end of the outer covering 376 can follow the contour of the outflow end 374 of the valve 354 (e.g., generally sinusoidal outflow end). The lobes 384 can be part of the outer sheath 376. Alternatively, the lobes 384 can be separate sheets of biocompatible (e.g., natural or synthetic) attached at the outflow end of the valve at the respective commissures 386. The lobes 384 thus provide extensions at the outflow end commissures 386, which can be secured to a patient's tissue (e.g., the patient's valve wall).

The prosthesis 350 also includes a flange 388 of a substantially soft and flexible material. The flange 388 extends radially outwardly from the implantation apparatus 352. In FIG. 11, the flange 388 is positioned an axially spaced apart location from the inflow end, namely adjacent the outflow end rim portion 362 of the apparatus 352. For instance, the flange 388 has a radially inner periphery or edge 390 that is attached (e.g., by sutures 392) to the outflow edge of the implantation apparatus 352. The radially inner edge 390 of the flange 388, for example, is attached at the outflow surface of the implantation apparatus 302 corresponding to the rim portion 360. This flange position is well suited for use in implanting the prosthesis at an aortic position or a pulmonic position of a patient's heart; although, the prosthesis 350 is not limited to use at such positions.

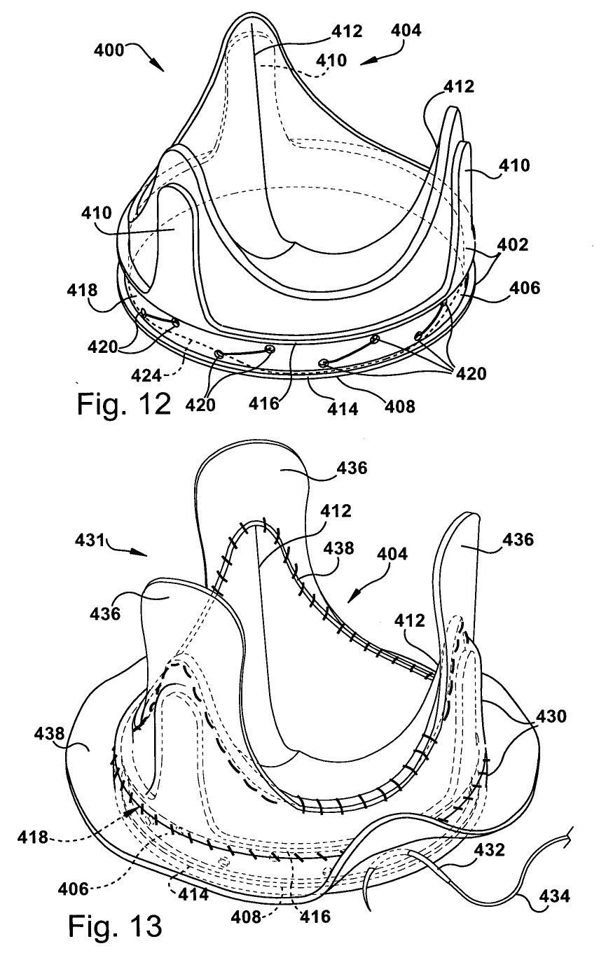

FIGS. 12 and 13 illustrate another example of a heart valve prosthesis 400 and 431 that can be implemented according to an aspect of the present invention. The prosthesis includes a support structure or stent 402 and valve member 404. While the valve member 404 is depicted as a natural tissue animal valve, it is to be understood that the valve member could be a different type of natural tissue valve, a mechanical valve or a biomechanical valve.

In the example of FIGS. 12 and 13, the stent 402 includes a base portion 406 having an inflow end 408 and a plurality of stent posts 410 extending substantially axially from the stent base portion 406 to define a generally sinusoidal outflow end. In the example of FIG. 12, the stent posts 410 are substantially radially aligned with respective commissures (e.g., juncture between adjacent pairs of leaflets corresponding to sinusoidal peaks or posts) 412 of the heart valve 404. The width of each of the respective stent post in the example of FIG. 11 are configured to be narrower than the remaining portions of sidewall of the natural tissue valve at the commissures 412. It is to be understood and appreciated, however, that other configurations and types of valves could be utilized with such stent 402.

The stent base portion 406 also includes a circumferentially extending channel (or channel) along a radially outer sidewall portion of the stent base portion. In FIG. 12, the channel is defined by the outer sidewall portion 418 of the stent base portion 406 that extends between a pair of rim portions 414 and 416. The rim portions 414 and 416 correspond to protruding members that extend radially outwardly from axially opposed ends of the stent base portion 406. The rim portions 414 and 416 thus correspond to structure, which can be either an integral part of the stent base portion (e.g., monolithic or single piece construction) to extend radially outwardly a distance that is greater than the at least a portion of the sidewall portion 418 that extends between the respective rim portions. As one example, the stent 402 is formed as a monolithic structure. Alternatively, the rim portions 416 and 414 can be attached to the stent base portion 406, such as by welding, adhesives, sutures or other attachment means.

The sidewall portion 418 of the stent base portion 406 can also include a plurality of apertures 420 extending through the sidewall portion 418. The heart valve 404 thus can be attached to the stent 402 via one or more sutures 422 to maintain the stent 404 at a substantially fixed position relative to the stent 402. The stent 402 can be positioned such that the inflow end 424 of the valve 404 is substantially coextensive (e.g., flush) with the inflow end 408 of the stent 402. Alternatively, the inflow end of the valve 404 may extend axially beyond the inflow end 408 of the stent 404.

As shown in FIG. 13 the prosthesis 400 of FIG. 12 can include a covering 430 of a substantially biocompatible material, such as described herein, to provide a covered prosthesis 431. The biocompatible material can include one or more sheets, a coating or other type of covering that is attached over at least a substantial portion of the prosthesis 400 (e.g., by sutures). As one example, the covering 430 can provide an exteriorized covering that completely covers the radially exterior surface of the stent 402 and helps to secure the stent to the valve 404. The covering 430 extends over the radially outer portion of the stent base portion 406 so that a chamber is formed by the channel 418 and the covering. The chamber extends circumferentially about the base portion 406. For instance, the chamber can be defined by adjacent surfaces of the covering 430, the sidewall portion 418 and adjacent surfaces of the rim portions 414 and 416. The chamber may be substantially hollow or, alternatively, the chamber may contain a substantially soft or flexible (e.g., natural or synthetic) biocompatible material, such as described herein.

By covering the annular channel along the sidewall portion 418 with the outer covering 430, implantation is facilitated. For instance, a needle 432 and a length of suture (indicated at 434) can be inserted through the outer covering 430 and into the channel in the sidewall portion 418 for securing the covered prosthesis 431 at a desired implantation site, such as via one or more purse string sutures. Thus, as the suture 434 is tightened around the annular channel in the sidewall portion 418, movement of the covered prosthesis 431 will be mitigated.

The covered prosthesis 431 may also include outflow lobes (or extension) 436 located at an outflow end 438 of the valve 404. For instance, the respective lobes 436 can be located at the radially outer surface near each of the commissures 412 between adjacent pairs of leaflets. The lobes 436 thus extend a predetermined distance axially beyond and lateral to each of the commissures 412 at the outflow end 438 of the valve 404. As one example, a portion of the outer covering 430 can extend axially beyond and laterally from the commissures 412 of the valve 404 to define the respective of lobes or extensions 436. Alternatively, sheets of substantially biocompatible material can be connected to the radial exterior of the valve posts to provide the lobes 436.

The covered prosthesis 431 can also include a flange 438 that extends radially outwardly from the base portion 406 of the prosthesis. In the example of FIG. 13, the flange 438 extends radially outwardly from the inflow end 408 of the base portion 406. The flange 438 can be attached to the outer covering 430 as well as attached to the inflow end 424 of the valve 404, such as by one or more sutures. The flange 438 extends radially outwardly from the prosthesis 400 a distance that is greater than the radially outward extent provided by the rim portions 414 and 416. After the prosthesis 431 has been implanted, such as by purse string suture applied around the channel in the sidewall portion 418, the flange 438 can be further attached at the valve annulus. The use of the flange 438 can also help improve hemodynamics for the prosthesis 400. The flange 438 can be formed of the same or different material from material utilized as the outer covering 430.

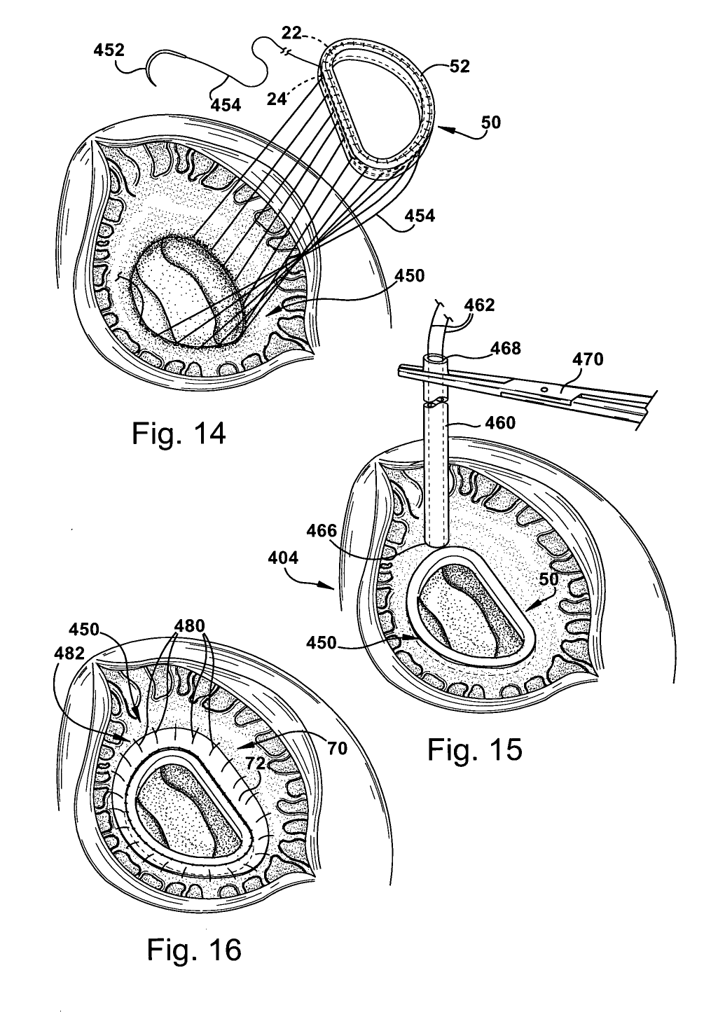

In view of the foregoing description of various apparatuses and prostheses that can be implemented according to an aspect of the present invention, implantation of such apparatuses will be better appreciated with reference to FIGS. 14-21. In the following FIGS. 14-21, it shown that a purse string suture can be implantation site. By utilizing a purse string suture, the implantation of these and other prosthesis can be facilitated according to an aspect of the invention. It is further to be appreciated that the implantation for each of the types of prosthesis described herein is substantially similar in that they involve the use of a purse string suture. For example, a single purse string suture can be applied at the annulus corresponding to the implantation site and around a base portion of the apparatus or prosthesis and tightened to thereby hold the apparatus or prosthesis relative to the implantation. If the apparatus includes an inflow flange, the flange can be extended outwardly from the apparatus or prosthesis to cover the juncture between the patient's tissue and the apparatus or prosthesis. By securing the flange to the surrounding tissue, the apparatus or prosthesis can be further secured as well as improve hemodynamics associated with blood flow through the apparatus or prosthesis.

FIGS. 14 and 15 depict purse-string implantation of the ring apparatus 50 of FIG. 2 at a heart valve annulus 450 according to an aspect of the present invention. The purse-string implantation employs a needle 452 and a length of a substantially biocompatible suture material 454. The suture can be a filament of a desired length, thickness and type of material as are readily available and known in the art. The needle 452 and suture 454 are passed via the annulus 450 at the desired implantation site and through the outer covering 52 at the channel 16.

As an example, the needle 402 can be passed alternatingly through the annulus 450 and through the outer covering 52 of the apparatus 50 overlying the channel 16, such as by taking predetermined size “bites” of each. The length or size of a given bite corresponds to the lateral distance between entry and exit sites of the needle, which results in a length of suture remaining in the object where the bite is taken between the entry and exit sites. The particular size of the bites in each of the annulus 450 and the apparatus 50 can vary according to the size of the patient and the size of the annulus 450 in which the device is being implanted. Typically, larger bites will be taken in the annulus 450, although the same or different sized bits can be used. By way of further example, the needle 452 can be passed via the annulus 450 at the implantation site with approximately 9 to 10 millimeter bite and by passing smaller bites through the covering 52 on the ring apparatus 50.

As an alternative, the purse string suture 454 need not be passed through the covering of the apparatus 50. For instance, the uncovered apparatus 10 of FIG. 1 can be used or the purse string can be applied via bites at the annulus 450 only. In either case, a tightening of the purse string suture 454 around the covered apparatus 50 (or around the channel 16 of the uncovered apparatus 10 of FIG. 1), will still enable the purse string suture to tighten sufficiently and remain seated in the channel 16 between the rim portions 22 and 24.

As shown in FIG. 15, after the purse string suture 454 has been applied in an appropriate manner along the annulus at which the ring 50 is to be implanted, such as described above, the purse string suture 454 may be tightened. For example, the remaining length of the suture 454 can be passed through an elongated cylindrical guide (or tube) 460 after exteriorization of the sutures, and the needle(s) can be cut out. As shown in FIG. 15, two lengths 462 of the suture 454 extend from the annulus 450 through the elongated cylindrical guide (or tube) 460. The guide 460 can be formed of a generally rigid material, such as rubber, plastic, or metal material. One end 466 of the guide 460 engages the annulus 450 and the ring apparatus 50. The two lengths 462 of the suture 454 extend through a central bore (or lumen) of the guide 460 and out the other end 468 of the guide.

A desired amount of tension can be applied to pull the two lengths 462 of suture through the guide 460 and urge the annulus 450 radially inwardly into desired engagement with the radial outer surface the apparatus 50. The two lengths 462 of suture can further be fixed relative to the guide 460, such as by clamping the sutures to the guide by a clamp 470. By further tightening the purse string 454 (by pulling the lengths 462 through the guide 460 while the guide is urged toward the annulus and the apparatus 50), the purse string suture can lie between rim portions 18 and 20 (e.g., substantially within the circumferential channel 16) so as to mitigate movement of the apparatus 50 relative to the annulus 450. The lengths 462 of suture 454 can be tied outside the heart near the end 468 of the tube (which can be exteriorized from the heart).

FIG. 16 depicts the ring apparatus 70 of FIG. 3 implanted at an annulus 450 of a patient's heart. The ring 70 can be attached at the annulus via one or more purse-string sutures according to the approach shown and described with respect to FIGS. 13-14. Additionally, the apparatus 70 includes a flange 72 which extends over the peripheral juncture between the ring apparatus and the annulus 450. Thus, after the ring 70 has been secured at the annulus 450, the flange 72 can be positioned so as to cover the juncture and any exposed sutures and then connected in place. For instance, one or more continuous sutures 480 can be applied to attach the flange 72 to the adjacent tissue 482 at the inflow side of the apparatus, such as shown in FIG. 16. The attachment of the flange 72 to such tissue 482 helps to secure the apparatus 70 at the annulus 454. Additionally, the flange 72 provides a smooth transition for blood flow, resulting in improved hemodynamics.

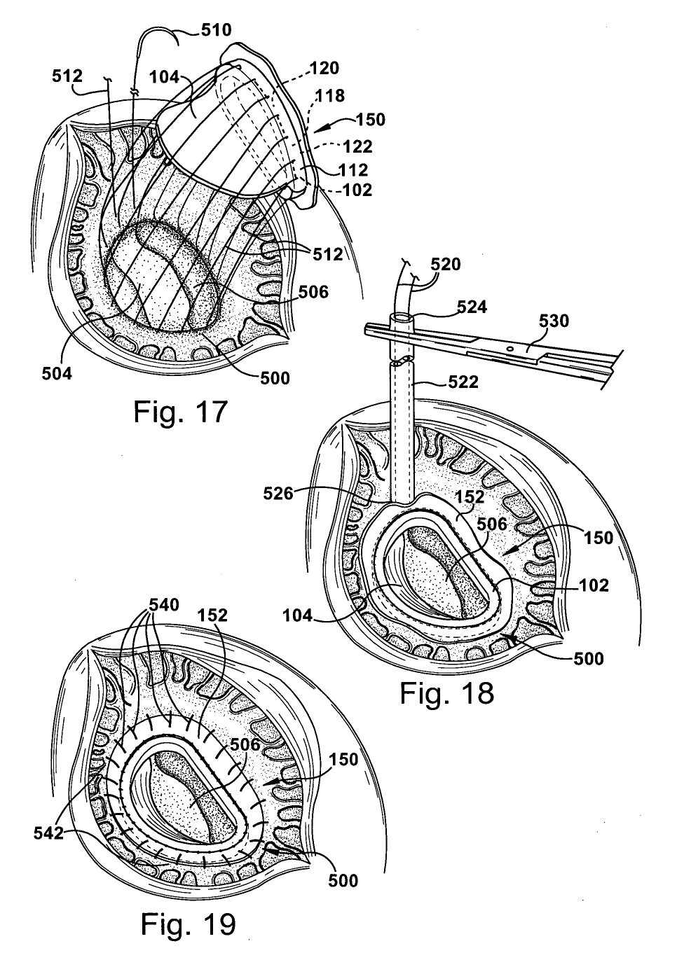

FIGS. 17, 18 and 19 depict portions of a procedure that can be utilized for implanting a valvuloplasty apparatus according to an aspect of the present invention. In the example of FIGS. 17-19, the apparatus corresponds to the apparatus 150 shown and described with respect to FIG. 5. Accordingly, reference may be made back to FIGS. 4 and 5 for additional information about the apparatus 150. As described herein, the apparatus 150 is configured to simulate part of a heart valve (e.g., one or more leaflets at closed position) to improve operation of a patient's existing valve. This type of device provides a desirable alternative in many circumstances to performing repair of the valve, such as a mitral valve repair.

The procedure for implanting the apparatus 150 is similar to the purse-string implantation procedure shown and described with respect to FIGS. 14-16. When implanting the apparatus 150 at an annulus 500 of the patient's heart valve 502, the buttress 104 is implanted at a desired angular orientation relative to the annulus. For instance, the buttress 104 is positioned in overlying relationship to the patient's defective leaflet (or leaflets) 504 and in a substantially diametrically opposed relationship relative to the one or more viable leaflets 506.

The purse-string implantation employs a needle 510 and a length of a substantially biocompatible suture material 512, such as described herein. The needle 510 and suture 512 are passed via the annulus 500 at the desired implantation site and through the outer covering 112 at the channel 118. As an example, alternating bites with the needle 510 can be passed through the annulus 500 and through the outer covering 112 overlying the channel 118. As an alternative example, the purse string suture 512 need not be passed through a covering of the apparatus 150 (e.g., a covering is not required). For instance, purse string sutures 512 can be applied via bites at the annulus 500 only and the purse string suture tightened around the base portion 102 between the axially opposed rim portions 120 and 122 to secure the apparatus 150 at the annulus 500.

After the purse string suture 512 has been applied in an appropriate manner along the annulus 500 at which the apparatus 150 is to be implanted, such as described above, the purse string suture may be tightened to secure the apparatus 150 at the annulus, as shown in FIG. 18. For example, the remaining length of the suture 512 can be passed as a two lengths 520 of suture through an elongated cylindrical guide (or tube) 522, such as described herein. A proximal end 524 of the guide 522 can be exteriorized relative to the heart, and then the sutures can be exteriorized through the guide, and the needle removed (e.g., by cutting).

The distal end 526 of the guide 522 thus engages the annulus 500 and the apparatus 150. The two lengths 520 of the suture extend through a central bore (or lumen) of the guide 522 and out the other end 524. A desired amount of tension can be applied to pull the two lengths 520 of suture proximally through the guide 522 while urging the guide distally into the annulus 500 and the base portion of the apparatus 150. This type of action causes the annulus 500 to move radially inwardly into desired engagement with the sidewall of the apparatus 150. The two lengths 520 of suture can further be fixed relative to the guide 522, such as by clamping the sutures to the cylinder using a clamp 530. The purse string suture thus lies within the channel 118 so as to mitigate movement of the apparatus relative to the annulus 500. The lengths 520 of the purse-string can be tied outside the heart near the end 524 of the guide to secure the apparatus at the annulus.

In FIG. 19, the flange 152 is positioned and secured over the peripheral juncture between the apparatus 150 and the annulus 500. Thus after the apparatus 150 has been secured at the annulus 500 via purse-string implantation (e.g., see FIGS. 17 and 18), the flange 152 can be positioned so as to cover the juncture and any exposed sutures and then be connected in place. For instance, one or more continuous sutures 540 can be applied to attach the flange 152 to the adjacent tissue 542 at the inflow side of the apparatus. The attachment of the flange 150 to such tissue 542 helps to further secure the apparatus 150 at the annulus 500. Additionally, the flange 150 provides a smooth transition for blood flow, resulting in improved hemodynamics.

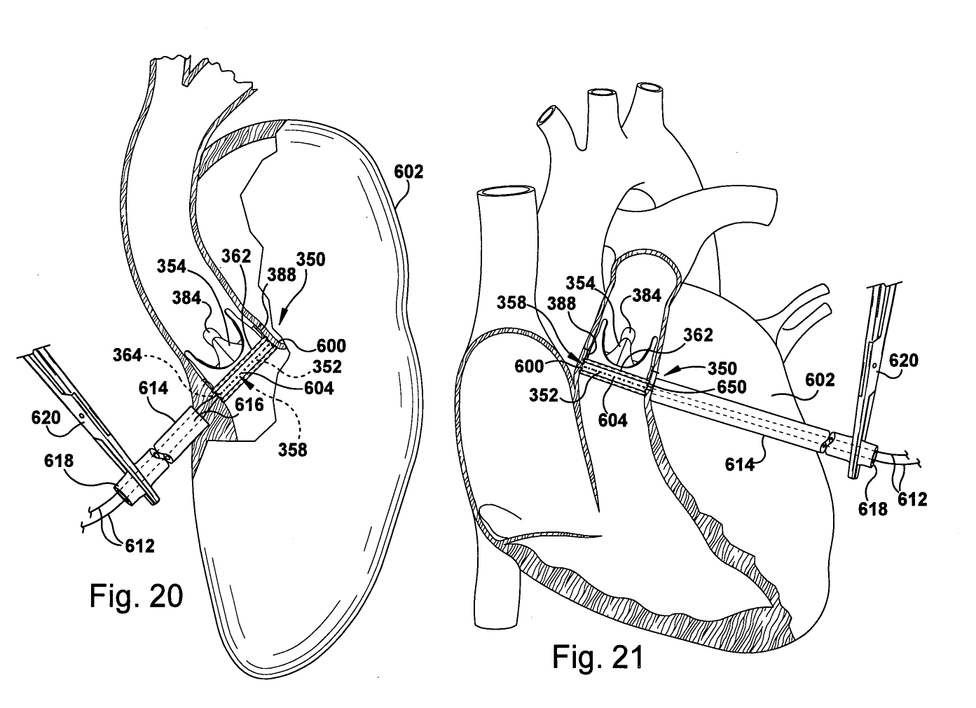

FIG. 20 depicts an example of the heart valve prosthesis 350 of FIG. 11 implanted at the aortic position of a heart 600, and FIG. 21 depicts the prosthesis 350 of FIG. 11 implanted at the pulmonic positions of the heart. It is to be understood and appreciated that any type of valve (e.g., natural tissue, mechanical or biomechanical) can be utilized for implantation based on the teachings contained herein. Additionally, the prosthesis can include an implantation apparatus of monolithic (FIG. 6, 7, 10 and 11) or a multi-piece construction (FIG. 9) or the channel might be part of the stent (e.g., FIGS. 12 and 13). It is further understood that the implantation apparatus (whether a single piece or multiple pieces attached together) can be formed of a sufficiently flexible and resilient material that it may be deformed during implantation to the implantation position and automatically return to its original, desired configuration.

Referring to FIG. 20, a typical implantation process for the prosthesis 350 at an aortic position will now be described. Prior to implanting the prosthesis 350, the patient's native valve or at least calcified portions thereof may be removed from the annulus 600 of the patient's heart 602. After appropriate preparations have been made and the prosthesis is near the implantation cite (the annulus 600), a purse-string suture 604 can be applied at the respective annulus 600 at which the heart valve prosthesis 350 is to be implanted. For the example of FIG. 20, the purse string suture 604 is applied at the aortic annulus 600. The purse string can be applied by taking alternating bites at the annulus 600 and the covering 326 overlying the sidewall 364 of the base portion (e.g., juxtaposed relative to the channel 358). The purse-string suture 604 initially can be sufficiently at loose such that the prosthesis 350 can be moved relative the annulus (see, e.g., FIGS. 14 or 17) 600 and then tightened (see, e.g., FIGS. 15 or 18).

By way of example, excess suture 604 can be passed as a two lengths 612 of suture through an elongated cylindrical guide (or tube) 614, such as described herein. A proximal end 618 of the guide 614 can be exteriorized relative to the heart, and then the sutures can be exteriorized through the guide, and the needle can be removed (e.g., by cutting). The distal end 616 of the guide 614 engages the annulus 600 and the outer sidewall 364 of the base portion 352 of the prosthesis 350 at a location from where the two lengths 612 of purse string suture extend. The two lengths 612 of the suture extend through a central bore (or lumen) of the guide 614 and out the proximal end 618. After the position of the prosthesis 350 has been confirmed as correct, a desired amount of tension can be applied to pull the two lengths 612 of suture through the guide 614 while the end 616 is held against the annulus 600 and the base portion 352. This action urges the annulus 600 radially inwardly into desired engagement with the sidewall of the base portion 352, such as within the channel 358 (e.g., similar to as shown and described with respect to FIGS. 15 and 18). The two lengths 612 of suture can further be fixed relative to the guide 614, such as by clamping the sutures to the cylinder using a clamp 620.

Thus, it will be understood and appreciated that a single purse-string suture should be sufficient to maintain the desired axial position of the heart valve prosthesis. By passing the suture filament through the outer covering of the implantation apparatus, the annular attachment is further augmented to mitigate angular (or rotational) movement of the prosthesis. After the purse string suture 604 has been tied off (e.g., through the guide), excess suture filament can be removed. The flange 380 then can be positioned over the juncture between the sidewall base portion and the annulus 600 and secured to surrounding tissue 610, such as by sutures. Additionally, lobes (if provided) can be employed to secure the commissures of the valve (or valve posts) to surrounding tissue in the aorta. By attaching the lobes 416 to the aortic wall, improved valve competence and coaptation can be achieved, and the likelihood of prolapse can be reduced.

FIG. 21 depicts a similar purse-string implantation of a heart valve prosthesis 350 at the pulmonic position. The implantation process can be similar to that described with respect to FIG. 20. In FIG. 21, the prosthesis 300 is implanted at a pulmonic annulus 650 of the patient's heart 602. Additionally, the flange 338 is located at the inflow end of the base portion 302, and thus is secured over the peripheral juncture between the pulmonic annulus 650 and the inflow end of the base portion 302. Those skilled in the art will understand and appreciate that the valve 350 as well as other types of valves shown and described herein may also be implanted for replacing a patient's aortic valve, a pulmonic valve, as well as for replacing a patient's atrioventricular valve (e.g., mitral valve or tricuspid valve). The particular type, size and configuration of prosthesis can be selected based on the intended implantation site and the patient's condition. The use of a purse-string, as shown and described herein, thus will facilitate implantation at any such implantation site.

In view of the foregoing, those skilled in the art will understand and appreciate that the apparatus and methods described herein enable a simplified approach for repair or replacement of a patient's heart valve. This approach employs a support portion that can be implanted using a single purse string suture to hold the apparatus at the desired implantation site such as an annulus of a patient's heart valve. By utilizing a purse string to implant an apparatus at the patient's heart, the implantation procedure is significantly simplified and expedited relative to many existing approaches. As a result, cardio pulmonary bi-pass can be greatly reduced reducing the associated complexity with the implantation procedure.

With reference back to FIGS. 14-21, in the case where the base portion includes no outer covering (e.g., the channel is exposed) or when the cardiac prosthesis is being implanted without passing the purse-string suture through the outer covering, the purse string may be tightened to reduce the annulus size to a diameter that is smaller than that of the inflow rim portion after the base portion has been inserted within the annulus at the desired implantation position. Once the prosthesis is positioned such that the annularized purse string circumscribes the channel of the implantation apparatus, the purse-string can be tightened to hold the apparatus. Additional sutures, such as multiple purse string sutures, or other connection means can optionally be used to secure the prosthesis at implantation site. One or more additional other connection means (clamps, hooks, barbs, and the like) can also be utilized for securing the prosthesis at the desired position relative an annulus as well as to other adjacent tissue proximal the annulus.

What has been described above includes examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. An implantable apparatus, comprising:
a substantially rigid and annular base portion having a radially outer sidewall portion and having a radially inner sidewall portion that defines an opening that extends axially through the base portion;
a first rim portion extending substantially radially outwardly from the radially outer sidewall portion; and
a second rim portion extending substantially radially outwardly from the radially outer sidewall portion at an axial location that is spaced apart from the first rim portion so as to define a substantially continuous annular channel that extends circumferentially along the radially outer sidewall portion intermediate the first rim portion and second rim portion.

2. The apparatus of claim 1, further comprising a covering of a substantially biocompatible material that covers at least the radially outer sidewall portion of the apparatus.

3. The apparatus of claim 2, wherein the covering completely covers the base portion, the first rim portion and the second rim portion.

4. The apparatus of claim 2, wherein the covering extends over the radially outer sidewall portion to provide a circumferentially extending chamber defined by the covering, the first rim portion, the second rim portion, and the radially outer sidewall portion intermediate the first and second rim portions.

5. The apparatus of claim 4, wherein the first and second rim portions are monolithic with the base portion and extend outwardly relative to the radially outer sidewall portion intermediate the first and second rim portions a distance that is sufficient to permit a surgical needle to take bites into the covering that overlies the channel.

6. The apparatus of claim 2, further comprising a flange of a substantially flexible and biocompatible material that extends radially outwardly from the base portion at location that is spaced axially apart from at least a portion of the channel.

7. The apparatus of claim 6, wherein the flange comprises at least one annular sheet of a substantially biocompatible material having an inner periphery that is attached to the covering adjacent one of the first and second rim portions.

8. The apparatus of claim 2, wherein the covering comprises a biological tissue material.

9. The apparatus of claim 1, further comprising a buttress member that extends substantially axially from the base portion over a portion of the opening, the buttress member having a radially inner surface that is dimensioned and configured to simulate the dimensions and configuration of at least one heart valve leaflet at a closed position.

10. The apparatus of claim 9, further comprising a covering of a substantially biocompatible material that covers at least the radially outer sidewall portion of the apparatus and surfaces of the buttress member.

11. The apparatus of claim 10, further comprising a flange of a substantially flexible and biocompatible material that extends radially outwardly and circumferentially from the base portion at a location that is spaced axially apart from at least a portion of the channel.

12. The apparatus of claim 1, further comprising a heart valve mounted within the opening of the base portion, the heart valve including at least one moveable member configured to permit substantially unidirectional flow of blood there through.

13. The apparatus of claim 12, further comprising a covering of a substantially biocompatible material that covers at least the radially outer sidewall portion of the apparatus and radially outer exposed surfaces of the heart valve.

14. The apparatus of claim 12, further comprising a flange of a substantially flexible and biocompatible material that extends radially outwardly and circumferentially from the base portion at a location that is spaced axially apart from at least a portion of the channel.

15. The apparatus of claim 14, wherein the radially inner sidewall portion extends axially between opposed inflow and outflow ends of the base portion, the flange extending radially outwardly from the base portion at an axial position adjacent one of inflow and outflow ends of the base portion.

16. The apparatus of claim 12, wherein the base portion is a base portion of a stent, the stent further comprising a plurality of stent posts that extend axially from an outflow end of the base portion, the heart valve comprising a natural tissue heart valve having commissures between adjacent pairs of leaflets, each of the commissures being substantially radially aligned with respective stent posts.

17. The apparatus of claim 16, further comprising a covering of a substantially biocompatible material that covers at least the radially outer sidewall portion of the apparatus and at least radially outer surfaces of the stent, the covering extending over the radially outer sidewall portion of the base portion to provide a circumferentially extending chamber defined by the covering, the first and second rim portions, and the radially outer sidewall portion intermediate the first and second rim portions.

18. The apparatus of claim 1, wherein each of the radially outer sidewall portion and the radially inner sidewall portion extends between axially opposed first and second end surface portions of the base portion, the first rim portion being defined, in part, by a radially outward extension of the first end surface portion, the second rim portion being defined, in part, by a radially outward extension of the second end surface portion.

19. The apparatus of claim 1, further comprising one of:
a buttress member that extends substantially axially from the base portion, the buttress member having a radially inner surface that is dimensioned and configured to simulate the dimensions and configuration of at least one heart valve leaflet at a closed position; or
a heart valve mounted within the base portion, the heart valve including at least one moveable member configured to permit substantially unidirectional flow of blood there through the base portion and the heart valve.

20. A cardiac prosthesis comprising:
an annular base portion having inflow and outflow end portions spaced apart by a substantially annular sidewall portion that is configured to maintain a circumferential length thereof, an aperture extending axially through the base portion;
a flexible covering of a substantially biocompatible material that connected over at least a radially outer surface of the sidewall portion; and
at least one feature that extends circumferentially around the sidewall portion and covered by the covering to form at least one corresponding chamber that is defined by the covering, the at least one feature and a portion the radially outer surface of the base portion.

21. The prosthesis of claim 20, wherein the at least one feature comprises a channel extending circumferentially in the sidewall portion of the base portion, the covering extending over the channel to define the chamber.

22. The prosthesis of claim 21, further comprising:
a first rim portion extending substantially radially outwardly from the radially outer surface of the sidewall portion; and
a second rim portion extending substantially radially outwardly from the radially outer surface of the portion at an axial location that is axially spaced apart from the first rim portion so as to define the channel intermediate the first rim portion and the second rim portion.

23. The prosthesis of claim 20, wherein the at least one feature is monolithic with the base portion.

24. The prosthesis of claim 20, further comprising a flange of a substantially flexible and biocompatible material that extends radially outwardly from the base portion adjacent one of the inflow or outflow end portions.

25. The prosthesis of claim 24, wherein the flange comprises at least one annular sheet of a substantially biocompatible material having an inner periphery that is attached to the covering adjacent one of the inflow or outflow end portions.

26. The prosthesis of claim 25, wherein the covering and the flange comprise a biological tissue material.

27. The prosthesis of claim 20, further comprising a buttress member that extends substantially axially from the base portion over a portion of the opening, the buttress member having a radially inner surface that is dimensioned and configured to simulate the dimensions and configuration of at least one heart valve leaflet at a closed position, the covering covers surfaces of the buttress member.

28. The prosthesis of claim 27, further comprising a flange of a substantially flexible and biocompatible material that extends radially outwardly and circumferentially from the base portion at a location that is adjacent one of the inflow or outflow end portions.

29. The prosthesis of claim 20, further comprising a heart valve mounted within the base portion, the heart valve including at least one moveable member configured to permit substantially unidirectional flow of blood there through, the covering covers at least radially outer exposed surfaces of the heart valve.

30. The prosthesis of claim 29, further comprising a flange of a substantially flexible and biocompatible material that extends radially outwardly and circumferentially from the base portion at a location that is adjacent one of the inflow or outflow end portions.

31. The prosthesis of claim 29, wherein the heart valve is one of a natural tissue heart valve, a mechanical heart valve, and a biomechanical heart valve.

32. The prosthesis of claim 29, wherein the base portion is a base portion of a stent, the stent further comprising a plurality of stent posts that extend axially from an outflow end of the base portion, the heart valve comprising a natural tissue heart valve having commissures between adjacent pairs of leaflets, each of the commissures being substantially radially aligned with respective stent posts.
